(19) United States
(12) Patent Application Publication
(10) Pub. No.: US 2007/0219457 A1
(43) Pub. Date: Sep. 20, 2007

(54) Wireless Ear-Clips Heart Rate Monitor

(76) Inventor: Chiu-Hsiang Lo, Taichung (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/459,867

(22) Filed: Jul. 25, 2006

(30) Foreign Application Priority Data
Mar. 17, 2006 (TW) 095204509

(51) Int. Cl. A61B 5/04

(52) U.S. Cl. 600/519

(57) ABSTRACT

This invention relates to a wireless ear-clips heart rate monitor comprising a sensor unit of ear-clips type that detects a user's heart rate, a signal-processing unit that receives and processes the signal generated from the sensor unit, and a wireless signal-transmitting unit that receives the signals from the signal-processing unit and then transmits the signals out. The sensor unit detects frequency of change of blood density to derive heart rate, with the features of precision and efficiency on the detection of heart rate, cooperating with the technique of wireless transmission, the purpose of promoting accuracy of detection and improving convenience of using is attained.

### FIELD OF THE INVENTION

This invention relates to a wireless heart rate monitor of ear-clips type comprising a sensor unit that detects frequency of change of blood density in a user's ear pinna and generates frequency signals, a signal-processing unit that receives and processes signals generated from the sensor unit, and a wireless signal-transmitting unit that receives signals from the signal-processing unit and then transmits the signals out for further application. With the features of precision and efficiency on the heart rate detection, cooperating with the technique of wireless transmission, the purpose of promoting accuracy of detection and improving convenience of using is attained.

### BACKGROUND OF THE INVENTION

As per the general recognition, a variety of heart rate monitors for detecting heart rate of a human body, monitoring and making use of the heart rate information are in the market; some of those are of the type being attached on body surface near the heart position of a human body to detect frequency of heart throb; another of those are of the type being fixed on body parts where pulsation is detectable, such as arms, wrists or thighs, to detect pulse rate; the other of those are of the ear-clips type comprising a light-emitting element, a light-receiving element and an ear clip that can be clipped on a user's ear pinna, such that the heart rate of a human body is derived by detecting the frequency of change of the blood density in the blood capillary of the heart systole (Refer similar technique to U.S. Pat. No. 6,223,069, in prior of U.S. Pat. No. 6,783,051). The above-mentioned heart rate monitor of detecting vibration frequency has to be attached on body surface near the heart position of a human body, it is inevitable to open up the user's clothes or to stretch out inside the user's clothes for applying the detector on the user, which is inconvenient and easy to get the user sick at his/her chest. Because of the motion of arm swinging (especially in status of exercising), the monitor that being fixed on arm or wrist to detect pulse rate is easy to get interfered with other objects or unable to detect continuously and precisely because of the shifting of the detection locations. Although the generally recognized ear-clips monitors have the features of easy wearing, convenience of using and precise detection of steady detection locations, connection wires are in the design of those monitors, which will cause inconvenience of being interfering with other objects.

The main technical concept of this invention is based on the advantages of ear-clips-monitors of easy wearing and precise detection of steady detection locations. With add-on design of wireless transmission providing function of wireless transmission for more convenience of using, further design and application have been extended.

Although the wireless transmission technique has been applied on the heart rate monitors of detecting frequency of heart throb or pulse rate, of which patents might have been acquired, patent application on the ear-clips-monitor has never been seen developed. With continuous research and testing, this invention of practicality and commercialization has been completed.

### SUMMARY OF THE INVENTION

The main intention of this invention is to provide a wireless heart rate monitor of detecting frequency of change of blood density in a human body, as well as wireless signal transmission, for promoting accuracy of detection and improving convenience of using. This invention comprises a sensor unit of ear-clips type, a signal-processing unit and a wireless signal-transmitting unit; the sensor unit contains a pair of clip arms for fixing on the user's ear pinna, having at least one of the two clip arms implemented with a light-emitting unit and a light-receiving unit, the frequency of blood density in the user's ear pinna is detected and a frequency signal is generated; the signal-processing unit receives and processes the frequency signal generated from the sensor unit, and the wireless signal-transmitting unit receives signals from the signal-processing unit and then transmits the signals out.

The second intention of this invention is to provide an easy-to-use wireless heart rate monitor that having the signal-processing unit and the wireless signal-transmitting unit integrated into a casing separated with the sensor unit, a connection wire being connecting said signal-processing unit and said sensor unit, the weight of the heart rate monitor is lessened for the user to wear in comfort.

The third invention of this invention is to provide an easy-to-wear wireless heart rate monitor that having the signal-processing unit and the wireless signal-transmitting unit integrated into a casing separated with the sensor unit, a deformable connection wire being connecting said signal-processing unit and said sensor unit, with the deforming and forming of the connection wire, a user can put the heart rate monitor on his/her ear fit.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a diagram showing the first embodiment of the present invention.

FIG. 2 is a diagram the system structure of required units based on the technique of IR (infrared) or RF (radio frequency);

FIG. 3 is a diagram showing the system structure of required units based on the technique of bluetooth;

FIG. 4 is a diagram showing the sensor unit being clipped on a user's ear pinna;

FIG. 5 is a diagram showing the first method of using of the first embodiment;

FIG. 6 is a diagram showing the second method of using of the first embodiment;

FIG. 7 is a diagram showing the first improved embodiment of the first embodiment;

FIG. 8 is a diagram showing the connection wire being bent and deformed referring to FIG. 7;

FIG. 9 is a diagram showing the deformed connection wire being worn on a user's ear;

FIG. 10 is a diagram showing the second improved embodiment of the first embodiment;

FIG. 11 is a diagram showing the deformed connection wire being worn on a user's ear according to FIG. 10, and

FIG. 12 is a diagram showing the second embodiment of this invention and the application.

### DETAILED DESCRIPTION OF THE PREFERRED EMBODIMENT

The major technique applied to this invention is specifically a design and application of the ear-clips-monitor operable to generate heart rate data of the user by realizing the theory of detecting the frequency of change of the blood density in the user's ear pinna, by which the heart rate is detected efficiently and the heart rate signal is transmitted out wirelessly, thereof operation is convenient on all needs such as heart rate monitoring during exercise or taking care of a patient.

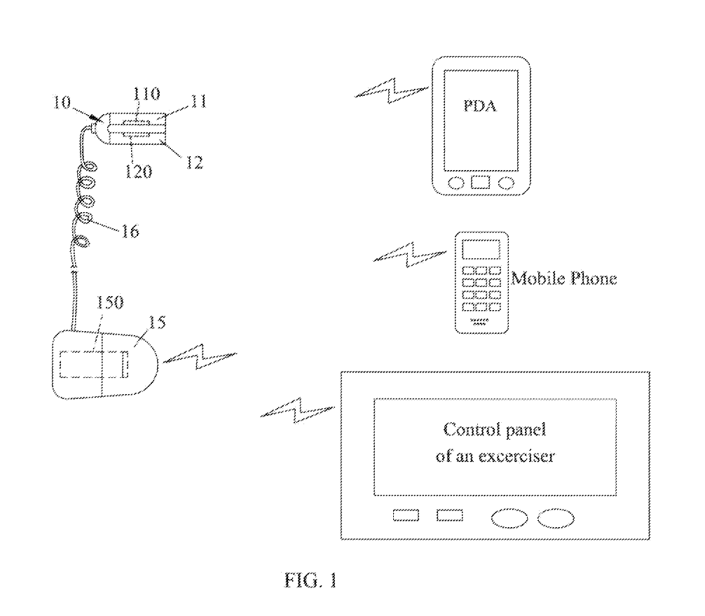

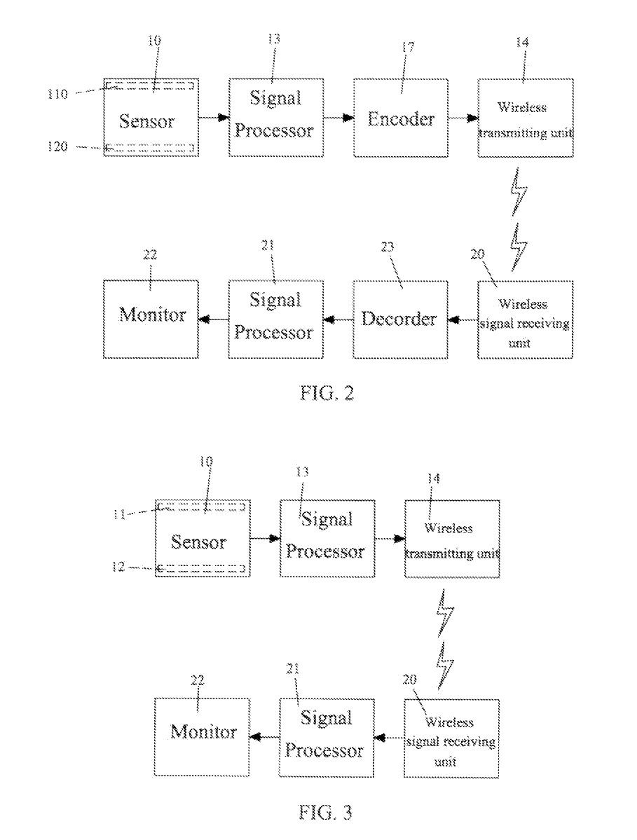

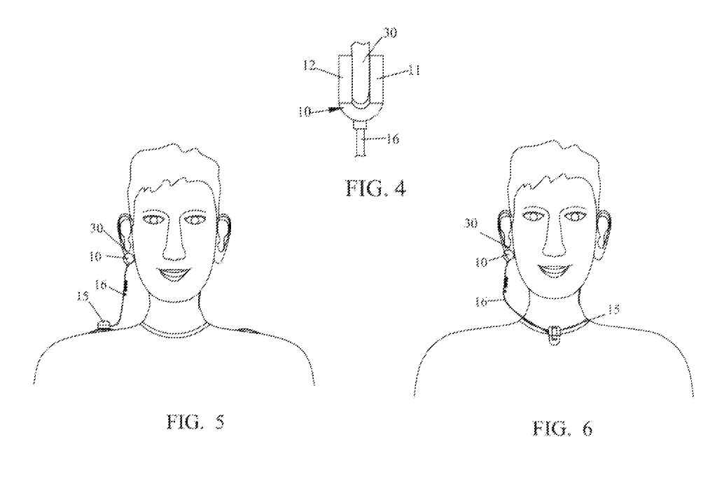

As shown on FIGS. 1 to 6, the first embodiment of this invention is structured basically as comprising a heart rate sensor unit 10, in which a pair of clip arms 11, 12 being contained for fixing on the user's ear pinna 30, at least one of the two clip arms 11, 12 having a light-emitting unit and a light-receiving unit implemented (not shown on the figures), an improved embodiment of this invention being utilizing an infrared-emitting unit 110 which being implemented on one clip arm 11 as the light-emitting unit, an infrared-receiving unit 120 being implemented on the other one clip arm 12 as the light-receiving unit, with which the frequency of change of the blood density in the blood capillary of a user's ear pinna 30 being detected and then a frequency data being generated, a signal amplifier being added to intensify the signal received on said infrared-receiving unit, and then transmitting the signal to the afterward mentioned signal-processing unit 13; a signal-processing unit 13 for processing the received frequency signal; a wireless signal-transmitting unit 14 for transmitting out the processed frequency signal. The aforementioned sensor unit 10 can efficiently detect heart rate of a user, with the use of the signal-processing unit 13 and the wireless signal-processing unit 14, the heart rate signal would be transmitted out wirelessly for application, implementing the cooperating wireless signal-receiving unit such as a PDA, a mobile phone or a control panel of an exercise machine, the received signal can be received and made use of. Practically the method of signal receiving and processing is to implement a wireless signal-receiving unit 20, a secondary signal-processing unit 21 and a display unit 22 on exercise machine, PDA or mobile phone, of which said wireless signal-receiving unit 20 can be infrared wireless signal-receiving unit, or an radio frequency wireless signal-receiving unit, or a bluetooth wireless signal-receiving unit; a decoder 23 should be added for the infrared wireless signal-receiving unit or the radio frequency wireless signal-receiving unit being employed. (As shown on FIG. 2)

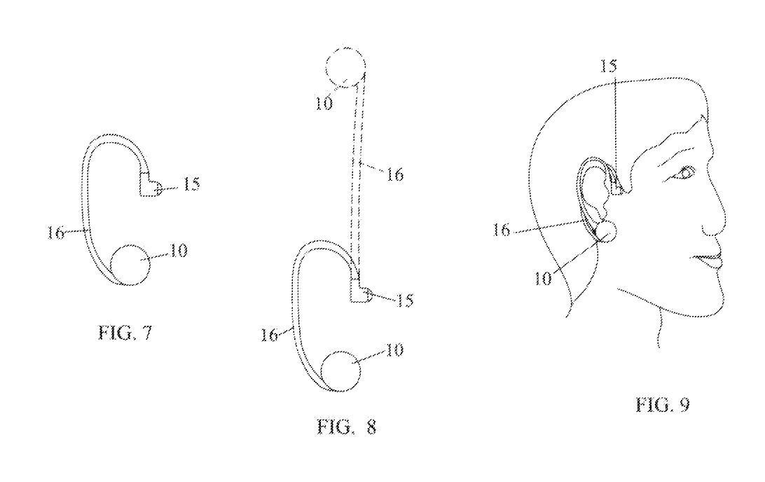

As shown on FIGS. 1 to 6, the first embodiment of this invention has the signal-processing unit 13 and the wireless signal-transmitting unit 14 integrated into a casing 15 separated with the sensor unit 10, a connection wire 16 being connecting said signal-processing unit 13 and said sensor unit 10. Referring to FIG. 1 for the first improved embodiment of the aforementioned, the clip 150 being implemented in the casing 15 and used to fix said casing 15 on the user's clothes (at side or front of the collar of the clothes, as shown on FIGS. 5 and 6), a user can put the casing 15 on the place where desired for improving the convenience of wearing; referring to FIGS. 7, 8 and 9 for the second improved embodiment of the aforementioned first embodiment, the connection wire 6 can be bent to deform and shaped for having on the user's ear.

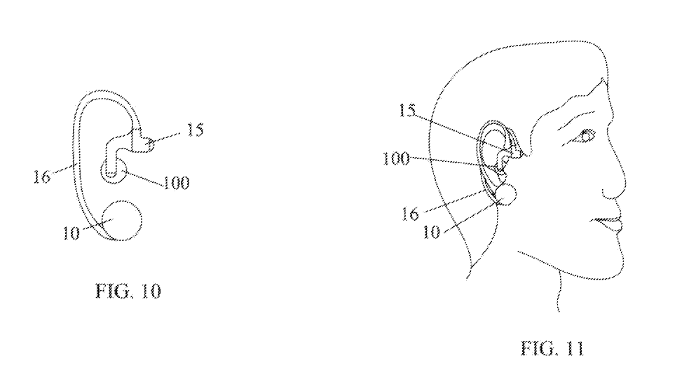

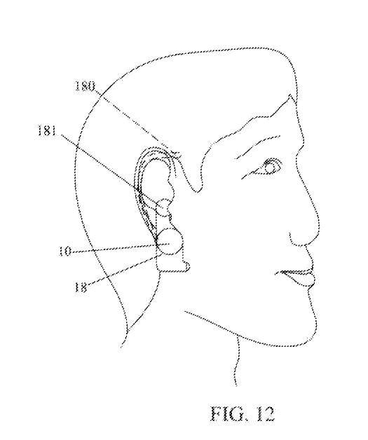

As shown on FIGS. 10 and 11, the second improved embodiment of the aforementioned first embodiment has a plug 100 extended from said sensor unit 10 for a user to put into his/her ear canal, in which a speaker (not shown on the figure) would be implemented to generate audio signal.

Besides, the design of this invention is done specifically on wireless signal transmission to an ear-clips heart rate monitor, of which several techniques of wireless transmission are applicable to the wireless signal-transmitting unit, such as infrared wireless transmission or radio frequency wireless transmission or bluetooth wireless transmission, in which infrared wireless transmission is the cheapest in cost. When infrared wireless transmission or radio frequency wireless transmission is utilized, signals should be decided, i.e. encoder should be provided. Structure of constituent of all units is as shown on FIG. 2, that is, connecting said signal-processing unit 13 with an encoder 17 and then to said infrared wireless transmitting unit or said radio-frequency wireless transmitting unit; when bluetooth wireless transmitting unit is utilized, there would be no need to do encoding to the signal but to connect said bluetooth wireless transmitting unit 14 with said signal-processing unit 13, as shown on FIG. 3.

As shown on FIG. 12, the second embodiment of this invention has the sensor unit 10, the signal-processing unit 13 and the wireless signal-transmitting unit 14 integrated into a casing 18, in which a hanging hook 180 being extended from the casing 18 for a user to wear on his/her lobe. Moreover, a plug 181 may be implemented extending from said casing 18 for a user to put into his/her ear canal, in which a speaker (not shown on the figure) would be implemented to generate audio signal.

While we have shown and described the embodiment in accordance with the present invention, it should be clear to those skilled in the art that further embodiment may be made without departing from the scope of the present invention.

What is claimed is:

1. A wireless heart rate monitor of ear-clips type comprising:
a heart rate sensor unit containing a pair of clip arms for fixing on a user's ear pinna, at least one of the two clip arms implemented with a light-emitting unit and a light-receiving unit, the frequency of blood density in the blood capillary of a user's ear pinna being detected and a frequency signal being generated;
a signal-processing unit receiving and processing signals generated from the sensor unit, and
a wireless signal-transmitting unit receiving signals from the signal-processing unit and then transmitting the signals out.

2. The monitor as claimed in claim 1, wherein the signal-processing unit and the wireless signal-transmitting unit are integrated into a casing which is separated with the sensor unit, said signal-processing unit and said sensor unit are connected with a connection wire.

3. The monitor as claimed in claim 2, wherein the connection wire can be bent to deform and shaped for hanging on the user's ear.

4. The monitor as claimed in claim 2, wherein the casing has a clip for a user to fix the heart rate monitor on the user's clothes, so the casing can be attached to the user.

5. The monitor as claimed in claim 2, wherein the sensor unit extends a plug for a user to put into the user's ear canal.

6. The monitor as claimed in claim 5, wherein the plug implements a speaker inside.

7. The monitor as claimed in claim 1, wherein the light-emitting unit is an infrared emitting unit, the light-receiving unit is an infrared receiving unit, a signal amplifier is added to intensify the signal received, the wireless signal-transmitting unit is a wireless infrared emitting unit, the signal-processing unit connects to an encoder and the encoder connects to the wireless infrared emitting unit.

8. The monitor as claimed in claim 1, wherein the wireless signal-transmitting unit is a wireless RF (radio frequency) emitting unit, the signal-processing unit connects to an encoder and the encoder connects to the wireless signal-transmitting unit.

9. The monitor as claimed in claim 1, wherein the wireless signal-transmitting unit is a wireless bluetooth RF (Radio Frequency) signal-emitting unit, the signal-processing unit connects to the wireless signal-transmitting unit directly.

10. The monitor as claimed in claim 1, wherein the sensor unit, the signal-processing unit and the wireless signal-transmitting unit are integrated into a casing.

11. The monitor as claimed in claim 10, wherein the casing extends a hanging hook for a user to wear in his/her lobe.

12. The monitor as claimed in claim 10, wherein the casing extends a plug for a user to put into his/her ear canal.

13. The monitor as claimed in claim 12, wherein the plug implements a speaker inside.

14. The monitor as claimed in claim 1, with further implements a wireless signal-receiving unit, a secondary signal-processing unit and a display unit.

15. The monitor as claimed in claim 14, wherein the wireless signal-receiving unit, the secondary signal-processing unit and the display unit are installed on a control panel of an exercise machine.

16. The monitor as claimed in claim 15, wherein the wireless signal-receiving unit is an infrared wireless signal-receiving unit with a decoder being connected.

17. A wireless heart rate monitor of ear-clips type comprising:
a heart rate sensor unit containing a pair of clip arms for fixing on the ear pinna of a user, at least one of the two clip arms implemented with an infrared emitting unit and an infrared receiving unit, the frequency of blood density in the blood capillary of a user's ear pinna being detected and a frequency signal being generated;
a signal-processing unit receiving and processing signals generated from the sensor unit;
a decoder connecting with the signal-processing unit, and
a wireless infrared signal-transmitting unit connecting with the decoder, by which signals being transmitted out;
the signal-processing unit, the decoder and the signal-transmitting unit are integrated into a casing separated with the sensor unit, the signal-processing unit and the sensor unit are connected with a connection wire.

18. The monitor as claimed in claim 17, wherein the connection wire can be bent to deform and shaped for hanging on the user's ear.

19. The monitor as claimed in claim 17, wherein the casing has a clip for a user to fix the heart rate monitor on the user's clothes, so the casing can be attached to the user.

20. A wireless heart rate monitor of ear-clips type comprising:
a heart rate sensor unit containing a pair of clip arms for fixing on a user's ear pinna, at least one of the two clip arms implemented with a light-emitting unit and a light-receiving unit, the frequency of blood density in the blood capillary of a user's ear pinna being detected and a frequency signal being generated;
a signal-processing unit receiving and processing signals generated from the sensor unit, and
a wireless signal-transmitting unit receiving signals from the signal-processing unit and then transmitting the signals out;
the sensor unit, the signal-processing unit and the signal-transmitting unit are integrated into a casing, in which a hanging hook being extended from the casing for a user to wear on his/her lobe.
